(19) United States
(12) Patent Application Publication
(10) Pub. No.: US 2019/0151673 A1
(43) Pub. Date: May 23, 2019

(54) Device for Electromagnetic Treatment of Topical Maladies and Associated Methods

(71) Applicant: Erchonia Corp., Melbourne, FL (US)

(72) Inventors: Steven Shanks, Melbourne, FL (US); Greg Bastin, Brighton (AU)

(73) Assignee: Erchonia Corp., Melbourne, FL (US)

(21) Appl. No.: 16/164,870

(22) Filed: Oct. 19, 2018

Related U.S. Application Data
(60) Provisional application No. 62/550,831, filed on Aug. 28, 2017.

(51) Int. Cl. A61N 5/06; A61M 37/00

(52) U.S. Cl. CPC A61N 5/0616; A61N 2005/0626; A61N 2005/0663; A61M 37/0015

(57) ABSTRACT

A device for electromagnetic treatment of maladies including a plurality of electromagnetic radiation (EMR) sources including a first EMR source operable to emit EMR having a peak emission wavelength of within a first wavelength range from 605 nanometers (nm) to 665 nm, a second EMR source operable to emit EMR having a peak emission wavelength within a second wavelength range from 502 nm to 562 nm, and a third EMR source operable to emit EMR having a peak emission wavelength within a third wavelength range from 375 nm to 435 nm. The device further includes a controller coupled to the plurality of EMR sources and operable to control the operation of each EMR source of the plurality of EMR sources responsive to an input and an input device coupled to the controller operable to conveying an input to the controller.

### RELATED APPLICATIONS

This application claims the benefit under 35 U.S.C. § 119(e) of U.S. Provisional Patent Application Ser. No. 62/550,831 titled Device for Electromagnetic Treatment of Topical Maladies and Associated Methods and filed on Aug. 28, 2017, the entire content of which is incorporated herein by reference

### FIELD OF THE INVENTION

The present invention relates to devices and methods for treating topical maladies by electromagnetic exposure.

### BACKGROUND OF THE INVENTION

Electromagnetic treatment of topical and sub-dermal maladies has traditionally been treated by the application of ointments, physical therapies, or sometimes irradiation by electromagnetic radiation within a single wavelength range. However, treatment using a series of differing electromagnetic radiation sources, each having a peak emission wavelength, is unknown in the industry. Accordingly, there is a need in the art for treatment methods utilizing three separate radiation sources having differing peak emission wavelengths and systems operable to perform such treatment methods.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a device for electromagnetic treatment of maladies comprising a plurality of electromagnetic radiation (EMR) sources comprising a first EMR source operable to emit EMR having a peak emission wavelength of within a first wavelength range from 605 nanometers (nm) to 665 nm, a second EMR source operable to emit EMR having a peak emission wavelength within a second wavelength range from 502 nm to 562 nm, and a third EMR source operable to emit EMR having a peak emission wavelength within a third wavelength range from 375 nm to 435 nm. The device may further comprise a controller coupled to the plurality of EMR sources and operable to control the operation of each EMR source of the plurality of EMR sources responsive to an input and an input device coupled to the controller operable to conveying an input to the controller.

The first wavelength range may be from 630 nm to 640 nm. The second wavelength range may be from 527 nm to 537 nm. The third wavelength range may be from 400 nm to 410 nm. The first EMR source may be a peak wavelength of 635 nm. The second EMR source may have a peak wavelength of 532 nm. The third EMR source may have a peak wavelength of 405 nm.

In some embodiments, the input device may be operable to receive a physical input from a user. In some embodiments, the input device may be operable to receive an input from a remote computerized device.

The controller may be operable to change at least one emission characteristics of the plurality of EMR sources selected from the group consisting of EMR intensity, waveform, waveform frequency, waveform amplitude, waveform pulse width, and waveform duty cycle.

In some embodiments, the device may further comprise a memory coupled to the controller and the input device and comprising a plurality of radiation treatment protocols. At least one of the input device and the controller may be operable to receive treatment information, defining received treatment information. The controller may be operable to select a radiation treatment protocol from the plurality of radiation treatment protocols responsive to the received treatment information, defining a selected radiation treatment protocol. Furthermore, the controller may be operable to operate the plurality of EMR sources responsive to the selected radiation treatment protocol.

In some embodiments, the treatment information comprises at least one of malady being treated, radiation dosage, and radiation emission characteristics.

Further embodiments of the inventions are directed to a method of treating a malady comprising emitting a first electromagnetic radiation (EMR) emission incident upon the topical malady having a peak emission wavelength of within a first wavelength range from 605 nanometers (nm) to 665 nm, emitting a second EMR emission incident upon the topical malady having a peak emission wavelength of within a second wavelength range from 502 nm to 562 nm, and emitting a third EMR emission incident upon the topical malady having a peak emission wavelength of within a third wavelength range from 375 nm to 435 nm.

In some embodiments, the first wavelength range is from 630 nm to 640 nm, the second wavelength range is from 527 nm to 537 nm, and the third wavelength range is from 300 nm to 410 nm. In some embodiments, the first EMR emission has a peak wavelength of 635 nm, the second EMR emission has a peak wavelength of 532 nm, and the third EMR emission has a peak wavelength of 405 nm.

In some embodiments, the method may further comprise performing a pre-irradiation procedure on tissue associated with the topical malady operable to alter the tissue. The pre-irradiation procedure may comprise at least one of injecting a solution, freezing, ablating, or micro-needling.

In some embodiments, the method may further comprise performing a post-irradiation procedure on tissue associated with the topical malady operable to alter the tissue. The post-irradiation procedure may comprise at least one of injecting a solution, freezing, ablating, or micro-needling.

In some embodiments, the method may further comprise receiving treatment information defined as a received treatment information and selecting a radiation treatment protocol responsive to the received treatment information defining a selected radiation treatment protocol. The first EMR emission, second EMR emission, and third EMR emission may be emitted having characteristics defined by the selected radiation treatment protocol. The treatment information may comprise at least one of malady being treated, radiation dosage, and radiation emission characteristics.

### BRIEF DESCRIPTION OF THE DRAWINGS

FIG. 1 is a schematic view of a system for providing electromagnetic treatment to a topical malady according to an embodiment of the invention.

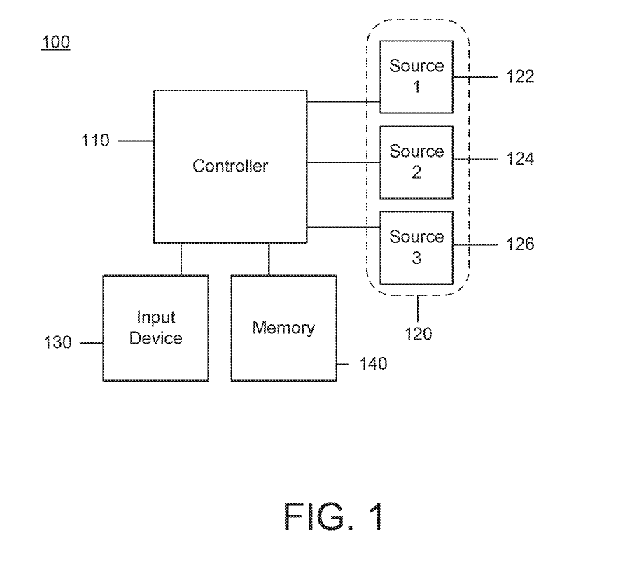

FIG. 2 is a flowchart illustrating a method of treating a topical malady according to an embodiment of the invention.

FIG. 3 is a flowchart illustrating a method of emitting electromagnetic radiation as part of the method of treatment of FIG. 2.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a device for emitting electromagnetic radiation for the treatment of topical maladies, including dermatological conditions such as verrucae (warts), cancerous growths, and others, as well as conditions causing chronic pain, including, but not limited to, arthritis, nerve damage, fibromyalgia, inflammatory myopathies such as dematomyositis, and the like.

A device 100 may comprise a controller 110, a plurality of electromagnetic radiation sources 120, an input device 130, and a memory 140 operably connected to the controller 110. The controller 110 may be operable to control the operation of the plurality of electromagnetic radiation sources 120. More specifically, the controller 110 may be operable to control the intensity of light emitted by one of, a subset of, or all the electromagnetic radiation sources comprised by the plurality of electromagnetic radiation sources 120. Moreover, the controller 110 may be operable to control the simultaneous operation of one of, a subset of, or all the electromagnetic radiation sources of the plurality of electromagnetic radiation sources 120. Additionally, the controller 110 may be operable to alter the emission characteristics of electromagnetic radiation emitted by the device, including, but not limited to, peak wavelength frequency, intensity, and modulation waveform including characteristics such as frequency, amplitude, pulse width, and duty cycle. Furthermore, the controller 110 may be operable to operate the plurality of electromagnetic radiation sources 120 to deliver sufficient radiation to affect a biological response in the tissue of a patient. In some embodiments, the controller 110 may be configured to determine a duration of operation of the plurality of electromagnetic radiation sources 120 to affect the desired biological response. Additionally, the controller 110 may be configured to operate the plurality of electromagnetic radiation sources 120 differently responsive to different desired biological responses. Such different desired biological responses may correspond to different maladies being treated by the device 100. The memory 140 may comprise a plurality of radiation treatment protocols, operable to provide instructions to the controller 110 that may then control the operation of the plurality of electromagnetic radiation sources 120 responsive to the instructions received from the memory. The controller 110 may be operable to select a protocol comprised by the memory 140 responsive to received treatment information, such as information indicating a desired radiation profile, a malady to be treated, and the like.

In the present embodiment, the plurality of electromagnetic radiation sources 120 comprises a first electromagnetic radiation source 122, a second electromagnetic radiation source 124, and a third electromagnetic radiation source 126. While three electromagnetic radiation sources are presented, any number of electromagnetic radiation sources is contemplated and included within the scope of the invention.

In some embodiments, the plurality of electromagnetic radiation sources 120 may comprise electromagnetic radiation sources having differing peak emission wavelengths. The peak emission wavelengths may be within the visible wavelength range of 400 nanometers (nm) to 760 nm or outside that range. The first electromagnetic radiation source 122 may have a peak emission wavelength within a range from 605 nm to 665 nm, or in some embodiments, within a range from 630 nm to 640 nm. The second electromagnetic radiation source 124 may have a peak emission wavelength within a range from 502 nm to 562 nm, or in some embodiments, within a range from 527 nm to 537 nm. The third electromagnetic radiation source 126 may have a peak emission wavelength within a range from 375 nm to 435 nm, or in some embodiments, 400 nm to 410 nm. In the present embodiment, the first electromagnetic radiation source 122 has a peak emission wavelength of 635 nm, the second electromagnetic radiation source 124 has a peak emission wavelength of 532 nm, and the third electromagnetic radiation source 126 has a peak emission wavelength of 405 nm. It is contemplated and included within the scope of the invention that the peak emission wavelength of the plurality of electromagnetic radiation sources 120 may vary by ±5 nm from the values provided herein.

The input device 130 may be any device capable of receiving an input and conveying that input to the controller 110. The controller 110 may be operable to operate the plurality of electromagnetic radiation sources responsive to the input received from the input device. In some embodiments, the input device 130 may be manipulated by the user to change one or more characteristics of electromagnetic radiation emitted by the device 100. Those characteristics include, but are not limited to, peak wavelength selection and combination, intensity selection, and modulating waveform characteristics frequency, amplitude, pulse width, and duty cycle.

The input device 130 may take many physical forms, including one or more dials, pushbuttons, switches, keypads, touchscreens, and the like. Moreover, in some embodiments, the input device 130 may be a communication device operable to receive an input from a remote computerized device. Such a communication device may be wired or wireless and comply with one or more communication standards, including, but not limited to, Ethernet, Universal Serial Bus, IEEE 802 standards, Bluetooth, Zigbee, Z-Wave, and any other communication standard as is known in the art. Additionally, the communication device may be operable to communicate across a network, including a personal area network (PAN), a local area network (LAN), and a wide area network (WAN), such as the internet.

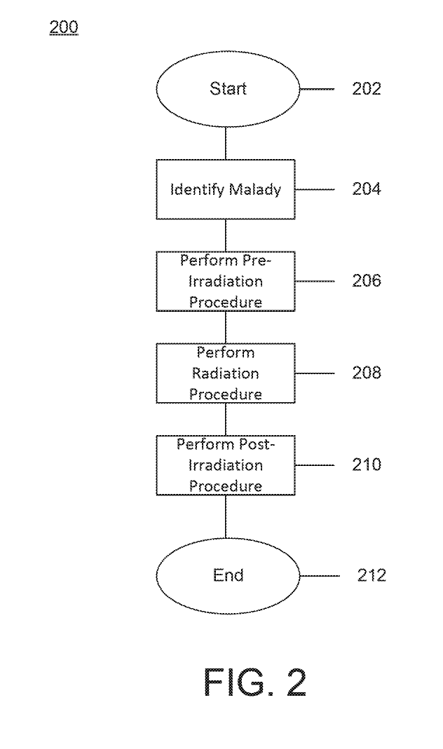

Referring now to FIG. 2, a flowchart illustrating a method 200 of treatment of a topical malady is presented. Beginning at 202, the method 200 may comprise identifying a topical malady for treatment 204. The method 200 may further comprise performing a pre-irradiation procedure 206 to the tissue associated with the malady. The pre-irradiation procedure may alter or damage the condition of the tissue associated with the malady. Such alteration or damage may promote an immune response by the body of the user. Such a pre-irradiation procedure may comprise treatments including, but is not limited to, injecting a solution, freezing, ablating (including, but not limited to, radio frequency, thermal, and electromagnetic radiation ablation), micro-needling, and the like.

The method 200 may further comprise performing the radiation procedure 208. Performance of the radiation procedure may comprise emitting electromagnetic radiation as described hereinabove.

The method 200 may further comprise performing a post-irradiation procedure 210. Like the pre-irradiation procedure, the post-irradiation procedure may alter or damage the condition of the tissue associated with the malady, and may comprise the same treatments as the pre-irradiation procedure. In some embodiments, the pre- and post-irradiation procedures may comprise the same treatments; in some embodiments, they may comprise differing treatments. The method 200 may end at 212.

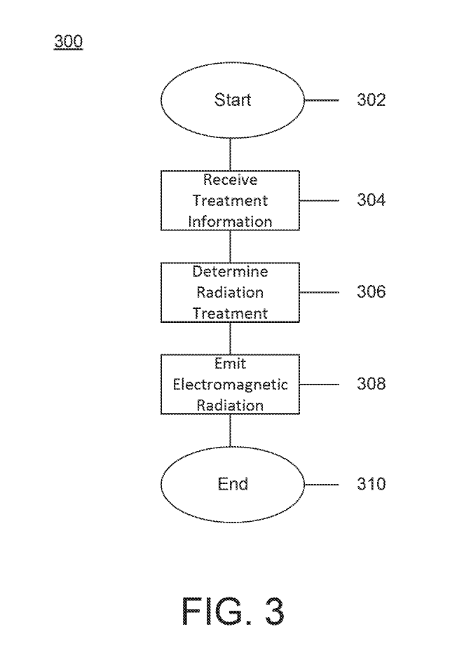

Referring now to FIG. 3, further characteristics of the method 200 are illustrated. Performing the radiation procedure at 208 may comprise the method 300 illustrated by the flowchart in FIG. 3. Starting at 302, the method 300 may comprise receiving treatment information 304. The treatment information received can vary and may include, but is not limited to, radiation dosage, malady being treated, radiation emission characteristics, and the like. Receipt of the treatment information may occur at a device operable to cause the emission of electromagnetic radiation responsive to the treatment information. In some embodiments, at least one of a controller operably coupled to a plurality of electromagnetic radiation sources or an input device operably connected to a controller may receive the treatment information.

The method 300 may further comprise determining a radiation treatment 306 responsive to the received treatment information. Determining a radiation treatment may comprise identifying the wavelengths of electromagnetic radiation to be emitted, determining the intensity of those wavelengths to be emitted, determining the treatment length for those identified wavelengths, determining a modulation waveform and its characteristics, and determining whether to emit the wavelengths simultaneously or sequentially. Such a determination may be made by identifying a treatment protocol identified as being therapeutically effective for the received treatment information.

The method 300 may further comprise emitting electromagnetic radiation 308 according to the radiation treatment determined at 306. The method 300 may then end at 310.

It is recognized that the above-described device and methods may also be effective in treating non-topical maladies, including subdermal maladies. Accordingly, nothing recited herein is intended to limit the scope of the invention solely to the treatment of topical maladies; the recitation of topical maladies is merely an exemplary embodiment. Treatment of subdermal maladies is contemplated and included within the scope of the invention.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

That which is claimed is:

1. A device for electromagnetic treatment of maladies comprising:
a plurality of electromagnetic radiation (EMR) sources comprising:
a first EMR source operable to emit EMR having a peak emission wavelength of within a first wavelength range from 605 nanometers (nm) to 665 nm;
a second EMR source operable to emit EMR having a peak emission wavelength within a second wavelength range from 502 nm to 562 nm; and
a third EMR source operable to emit EMR having a peak emission wavelength within a third wavelength range from 375 nm to 435 nm;
a controller coupled to the plurality of EMR sources and operable to control the operation of each EMR source of the plurality of EMR sources responsive to an input; and
an input device coupled to the controller operable to conveying an input to the controller.

2. The device of claim 1 wherein:
the first wavelength range is from 630 nm to 640 nm;
the second wavelength range is from 527 nm to 537 nm; and
the third wavelength range is from 400 nm to 410 nm.

3. The device of claim 1 wherein:
the first EMR source has a peak wavelength of 635 nm;
the second EMR source has a peak wavelength of 532 nm; and
the third EMR source has a peak wavelength of 405 nm.

4. The device of claim 1 wherein the input device is operable to receive a physical input from a user.

5. The device of claim 1 wherein the input device is operable to receive an input from a remote computerized device.

6. The device of claim 1 wherein the controller is operable to change at least one emission characteristics of the plurality of EMR sources selected from the group consisting of EMR intensity, waveform, waveform frequency, waveform amplitude, waveform pulse width, and waveform duty cycle.

7. The device of claim 1 further comprising a memory coupled to the controller and the input device and comprising a plurality of radiation treatment protocols;
wherein at least one of the input device and the controller is operable to receive treatment information, defining received treatment information;
wherein the controller is operable to select a radiation treatment protocol from the plurality of radiation treatment protocols responsive to the received treatment information, defining a selected radiation treatment protocol; and
wherein the controller is operable to operate the plurality of EMR sources responsive to the selected radiation treatment protocol.

8. The device of claim 7 wherein the treatment information comprises at least one of malady being treated, radiation dosage, and radiation emission characteristics.

9. A method of treating a malady comprising:
emitting a first electromagnetic radiation (EMR) emission incident upon a topical malady having a peak emission wavelength of within a first wavelength range from 605 nanometers (nm) to 665 nm;
emitting a second EMR emission incident upon the topical malady having a peak emission wavelength of within a second wavelength range from 502 nm to 562 nm; and
emitting a third EMR emission incident upon the topical malady having a peak emission wavelength of within a third wavelength range from 375 nm to 435 nm.

10. The method of treating a malady of claim 9 wherein the first EMR emission has a peak wavelength of 635 nm, the second EMR emission has a peak wavelength of 532 nm, and the third EMR emission has a peak wavelength of 405 nm.

11. The method of treating a malady of claim 9 wherein the first wavelength range is from 630 nm to 640 nm, the second wavelength range is from 527 nm to 537 nm, and the third wavelength range is from 300 nm to 410 nm.

12. The method of treating a malady of claim 9 further comprising performing a pre-irradiation procedure on tissue associated with the topical malady operable to alter the tissue.

13. The method of treating a malady of claim 12 wherein the pre-irradiation procedure comprises at least one of injecting a solution, freezing, ablating, or micro-needling.

14. The method of treating a malady of claim 9 further comprising performing a post-irradiation procedure on tissue associated with the topical malady operable to alter the tissue.

15. The method of treating a malady of claim 14 wherein the post-irradiation procedure comprises at least one of injecting a solution, freezing, ablating, or micro-needling.

16. The method of treating a malady of claim 9 further comprising:
receiving treatment information defined as a received treatment information; and
selecting a radiation treatment protocol responsive to the received treatment information defining a selected radiation treatment protocol;
wherein the first EMR emission, second EMR emission, and third EMR emission are emitted having characteristics defined by the selected radiation treatment protocol.

17. The method of treating a malady of claim 16 wherein the treatment information comprises at least one of malady being treated, radiation dosage, and radiation emission characteristics.

18. The method of treating a malady of claim 9 wherein the malady is one of topical and sub-dermal.

19. A method of treating a malady comprising:
receiving treatment information comprising at least one of the malady being treated, radiation dosage, and radiation emission characteristics, defined as a received treatment information; and
selecting a radiation treatment protocol responsive to the received treatment information defining a selected radiation treatment protocol;
emitting a first electromagnetic radiation (EMR) emission incident upon the topical malady having a peak emission wavelength of within a first wavelength range from 630 nanometers (nm) to 640 nm responsive to the selected radiation treatment protocol;
emitting a second EMR emission incident upon the topical malady having a peak emission wavelength of within a second wavelength range from 532 nm to 542 nm responsive to the selected radiation treatment protocol; and
emitting a third EMR emission incident upon the topical malady having a peak emission wavelength of within a third wavelength range from 400 nm to 410 nm responsive to the selected radiation treatment protocol.

20. The method of treating a malady of claim 19 further comprising performing at least one of a pre-irradiation procedure or a post-irradiation procedure on tissue associated with the topical malady operable to alter the tissue, the pre-irradiation procedure or the post-irradiating procedure comprising at least one of injecting a solution, freezing, ablating, or micro-needling.
